(19) United States
(12) Patent Application Publication
(10) Pub. No.: US 2026/0263095 A1
(43) Pub. Date: Sep. 10, 2026

(54) Pressure Wave Apparatus With Double Valve Means

(71) Applicant: STORZ MEDICAL AG, Tägerwilen (CH)

(72) Inventors: Rafael Storz, Kreuzlingen (CH); Markus Belau, Konstanz (DE); Arvid Kühl, Tägerwilen (CH); Lukas Honsell, Reichenau (DE); Felix Gremlich, Kreuzlingen (CH); Thomas Glenzer, Kreuzlingen (CH)

(21) Appl. No.: 19/105,754

(22) PCT Filed: Aug. 16, 2023
(86) PCT No.: PCT/EP2023/072556

§ 371 Date: Feb. 21, 2025

(30) Foreign Application Priority Data
Aug. 25, 2022 (EP) 22192131.5

(51) Int. Cl. A61B 17/22; A61B 17/00

(52) U.S. Cl. CPC A61B 17/22004; A61B 2017/00544

(57) ABSTRACT

The invention relates to an apparatus for treatment with pressure waves, comprising: a projectile (8) guided along a movement path, an applicator (7) at one end of the movement path, pneumatic means for application of pressure to the projectile (8) for the purpose of movement along the movement path, wherein the projectile (8) is adapted for striking onto the applicator (6) for generating the pressure waves, which pneumatic means comprises a double valve means (1, 2) for application of pressure to the projectile (8) towards the applicator (6) during a first activation time and in the reverse direction during a second activation time, and a control means (54) of the double valve means (1, 2), wherein the apparatus is adapted to allow the activation times to overlap.

The invention relates to an apparatus for treatment of the human or animal body with mechanical pressure waves generated by impact of an accelerated projectile onto an applicator.

Apparatus of this type have been known for some time and are increasingly in use. Mechanical pressure waves are used for treatment of the (human or animal) patient, which are coupled-in by placing an applicator onto the patient's body and are generated by (typically periodically repeated) collisions of an accelerated projectile with the applicator. The applicator does not necessarily have to be in one piece but can also be composed of a number of different parts or materials.

A technique for accelerating the projectile, which has been proven in practice and has been described many times, is pneumatic. A pneumatic overpressure is coupled-in by application of a pressure to a volume on one side of the projectile movable along a movement path, for example in a pipe segment.

In the prior art, a switching valve is used for this purpose, which is connected to a pneumatic supply, in particular a compressor with adjustable output pressure, and the pulse of which accelerates the projectile from an end of the movement path distal to the applicator towards the applicator. The pneumatic application is switched off when the proximal end of the movement path is reached, i.e., with the impact on the applicator.

In the prior art, the return movement takes place with the aid of a counter-pressure chamber, i.e., a storage volume, into which the projectile moved towards the applicator to a certain extent displaces the air located in front of it, whereby it virtually pumps up this storage volume.

In the prior document EP 2 181 730 B1, which, however, was revoked because of lack of reproducibility in opposition appeal proceedings, in addition to a control of the opening time of the switching valve for the acceleration, which is not explained in more detail, a pressure limitation in this counter-pressure chamber is also discussed. Alternatively, this document mentions the use of a second switching valve for a return of the projectile into the distal starting position after the application by the first switching valve.

The present invention is based on the object of specifying an apparatus of the described type with pneumatic means for projectile movement, which apparatus is improved with regard to the back and forth movement of the projectile.

In order to achieve this object, the apparatus according to claim 1 is proposed. Preferred configurations are the subject matter of the dependent claims.

Accordingly, the apparatus according to the invention has, as part of its pneumatic means, a double valve means for application of the projectile in both directions, i.e., towards the applicator and vice versa away from it in the reverse direction, i.e., for example the combination of a first and a second valve. This typically takes place repeatedly and iteratively in a sequence. The time phases in which the projectile is applied pneumatically in such a way that it moves in the forward direction, i.e., for example the activation phase of a first valve, is referred to below as the first activation time and vice versa as the second activation time a time phase of a reversed application of the projectile. According to the invention, the apparatus is to be adapted (i.e., in particular a control means present therein is to be adapted) in such a way that a second activation time already begins, while the first activation time still lasts, or vice versa. There is therefore an overlap time between the two valve opening times.

As a result, different advantages can be achieved in the individual case, depending on the requirements and the centers of gravity. In principle, the invention offers an additional degree of freedom with the overlap time, which can be used, for example, to control the impact speed of the projectile upon impact onto the applicator by changing the overlap time. Specifically, if, in this example, the second valve opening time already begins at a time before the collision during the first valve opening time, a counterforce acts on the projectile in addition to the pneumatic force accelerating in the forward direction. In the simplest case, when using approximately the same pneumatic pressure, this counterforce can be approximately equal in magnitude and virtually neutralize the acceleration. Depending on the magnitude of the portion of the first valve opening time before this time, the projectile is accelerated to a higher or lower speed, which it then approximately maintains, for example, during the overlap time until the collision.

In the opposite case, the like applies: if the overlap time arises as a result of the fact that, for example, the second valve opening time is not only used up to the maximum of the complete return of the projectile into the starting position, but also continues somewhat beyond this, but the first activation time already begins during the second activation time, no significant force again acts on the projectile during this overlap time (in the simplest case). Thus, there can be a phase without pneumatic forward acceleration of the projectile (possibly already during the return movement and after the end of the return movement), because pneumatic forward acceleration starts only after the end of the overlap time.

One advantage can consist in the fact that the impact speed of the projectile can be controlled more precisely and/or more easily than in the conventional comparison case, which was dependent on the opening time of the (single) switching valve and, of course, the effective pressure. Real switching valves actually have finite opening and closing times, i.e., they do not open and close instantaneously. This applies above all to the comparison between the closing time and the opening time, for example, in the case of (here preferred) spring-loaded valves, in which the opening process takes place magnetically and is optionally pneumatically assisted using the pressure to be switched, whereas the closing process takes place by a spring which is tensioned during opening. Experience has shown that there can be design-related and also ageing-related deviations and a different ageing behavior between the opening and closing times (here opening time in the sense of the opening process) can exist.

In the first above case of an overlap time at the end of a first valve opening time and at the beginning of a second valve opening time, the time period relevant to the projectile acceleration, namely the portion of the first valve opening time before this overlap time, is caused by the time difference between two valve opening processes (firstly of the first and then of the second valve). In the second above case, the overlap time is at the end of the second valve opening time. Here, the pneumatic projectile acceleration in the direction of the applicator becomes essential only at the end of the overlap time, so that the remaining portion of this first valve opening time after the overlap time and therefore the difference between two valve closing processes (firstly of the second and then of the first valve) is the relevant one. In both cases, this is therefore the time difference between similar valve movements.

The inventors have found that, in this manner, above all ageing-related deviations become significantly less noticeable (namely if, for example, the closing time exhibits stronger ageing influences than the opening time), because these influences are at least partially compensated by the described difference formation.

In the cited document from the prior art, on the other hand (with all conciseness of the representation relevant here), an alternating mode of operation of the two mentioned valves can be assumed, which also matches the particularly high intensities of the projectile-applicator collisions and therefore pressure waves aimed for in this document.

A further (alternative or additional) aspect can be to work, on the one hand, with a relatively high pneumatic pressure in order to achieve a rapid return and therefore also a high operating frequency, but, on the other hand, not necessarily having to use high impact speeds corresponding to this high pressure (when such an acceleration pressure is present during the entire forward movement). High intensities are not always desired for therapeutic reasons and are otherwise regularly connected with an increased stress on the patient due to pain or other irritations.

In order to avoid misunderstandings, it should be made clear that the term used here of the valve opening time principally includes both the temporal duration and the position relative to temporal reference points, in particular relative to the respectively other valve opening time. The term thus includes the start and the end of the valve opening time and the distance therebetween, unless expressly only the duration or only a start or end time is mentioned below.

The combination of two switching valves was addressed further above, which represents a possibility for a double valve means provided according to the invention. In this variant, the two valves can be controlled (preferably independently of one another) by the control means. Alternatively, however, a uniform valve can also be used, which is referred to here as a “combination valve” and which, depending on the control by the control means, has at least two switching states, namely a first for application of pneumatic pressure to the projectile in the direction towards the applicator and a second for application of pneumatic pressure to the projectile in the reverse direction. While the combination valve is in the first switching state, there is therefore a first valve opening time and, accordingly, a second valve opening time in the second switching state.

In these two switching states, the pneumatic connection to be applied in the respectively other switching state is preferably ventilated by the combination valve, so that, for example, during the forward movement, approximately ambient pressure prevails on the side of the projectile proximal to the applicator and, in contrast to the conventional procedure with a counter-pressure chamber, there is no dynamic pressure increasing from collision to collision.

However, the combination valve also has a further third switching state in which the two pneumatic connections are (simultaneously) applied with the pneumatic supply pressure. In this third switching state, there is therefore the overlap between the first activation time and the second activation time. If, therefore, the start of the second activation time during the first activation time (or vice versa) is mentioned above, this means, in the variant with the combination valve, a switching over or toggling of this combination valve. The same applies to an end of the first activation time during a still existing second activation time (or vice versa), i.e., an end of the overlap time.

Even when using two separate valves, at least one of the two valves is preferably a “two-way valve”, which accordingly carries out a ventilation, provided that it is not switched for application of the pneumatic pressure. However, further switching states are not excluded and the valve is not necessarily limited to precisely two switching states.

A ventilation is otherwise meant to mean a pneumatically highly conductive connection to the external atmosphere or to a reference pressure volume substantially corresponding thereto. It is therefore not a matter of a deliberate delay of the outflow of gas under overpressure in the sense of a throttling.

As an alternative to a ventilation via the combination valve or the two-way valves just addressed, the apparatus could also have, for example, a certain pneumatic leakage and, in the absence of an application of pneumatic pressure, carry out a throttled ventilation itself in this way or in a virtually creeping manner. However, this alternative is less preferred.

In the first case described above, namely an overlap time at the end of a first activation time, the second activation time and therefore the overlap time are started during a forward movement of the projectile. There is therefore a final phase of this forward movement, in which pressure is present on both sides of the projectile. This results in the possibilities already explained at the outset. However, this feature is not mandatory, since even (only) after the impact, that is to say during or at the start of the return movement of the projectile, a presence of pressure on both sides can be expedient. For example, the supply pressure for the return movement, which is desired because of the acceleration of the projectile, can be dimensioned to be somewhat excessive, for example because it is undesirable to allow the projectile to impact distally from the applicator with the same force as on the applicator. In this sense, for example, a simultaneousity of first and second valve opening time at the start of the return movement can have a certain throttling effect with regard to the return movement.

In the second case described above, namely an overlap time at the end of a second activation time, an effect similar to that just described can still be achieved with part of the overlap time during the return movement. The same also applies to such a simultaneousity toward the end of the return movement, that is to say before the next acceleration process or acceleration event.

In particular, the overlap time can be partly before and partly after the impact of the projectile on the applicator or else partly before and partly after the point furthest from the applicator has been reached, as will be explained in more detail in conjunction with the exemplary embodiment.

Furthermore, in the first case of the overlap time at the end of the first activation time, it is preferred to end the first activation time during the second activation time and thus to allow the second activation time to typically continue beyond the first activation time. In particular, this of course applies to the described return of the projectile. However, this feature is also not mandatory. For example, the described compensation of the accelerating force by a simultaneousity of first and second activation time (that is to say application of pneumatic pressure on both sides of the projectile) can also take place completely within a first activation time by a comparatively short second activation time. The projectile can then be returned, for example, in a manner already known from the prior art. Principally, even a further second activation time (which is separate from the second activation time during the first) could be used for the return.

Analogously, in the second case of an overlap time at the end of the second activation time, it is also preferred to allow the second activation time to end during the (next) first activation time, in particular in order therefore to bring about the already described pneumatic acceleration process of the projectile in the direction of the applicator.

If, as is preferred, an overlap time or portion of an overlap time is also present at least during the forward movement (that is to say before the impact), the portion of the first activation time before or after this overlap time (depending on the case under consideration) is preferably used to control the impact speed. In this case, in a simple case, for example, a finite, perhaps also only small number of different discrete such portions of the first activation time can be provided and can be set by the control means, in the simplest case only two. This includes the case in which an overlap time zero exists in one of the two control states (or more generally in a part of the control states).

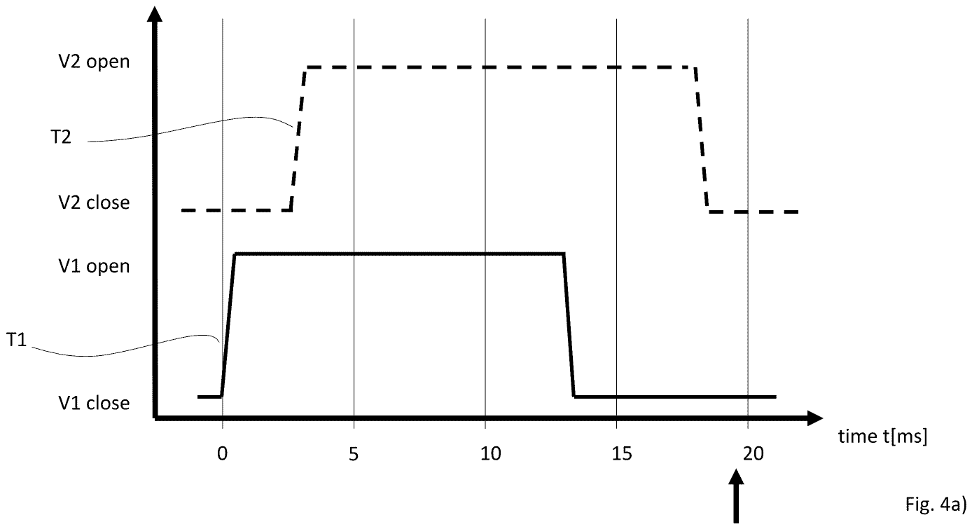

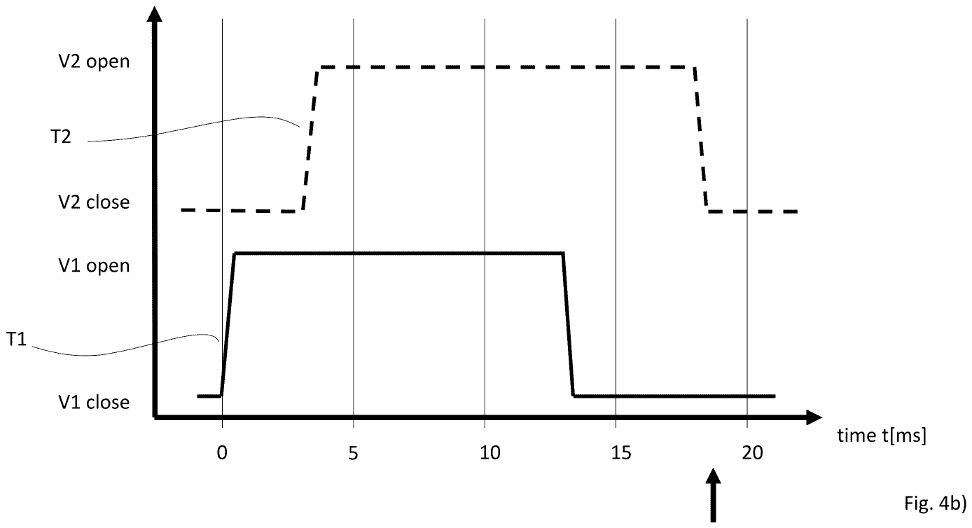

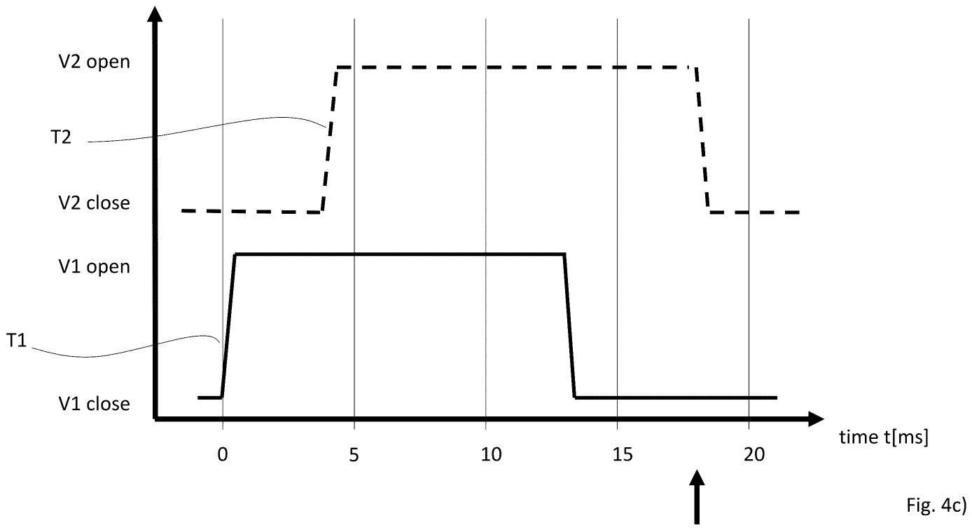

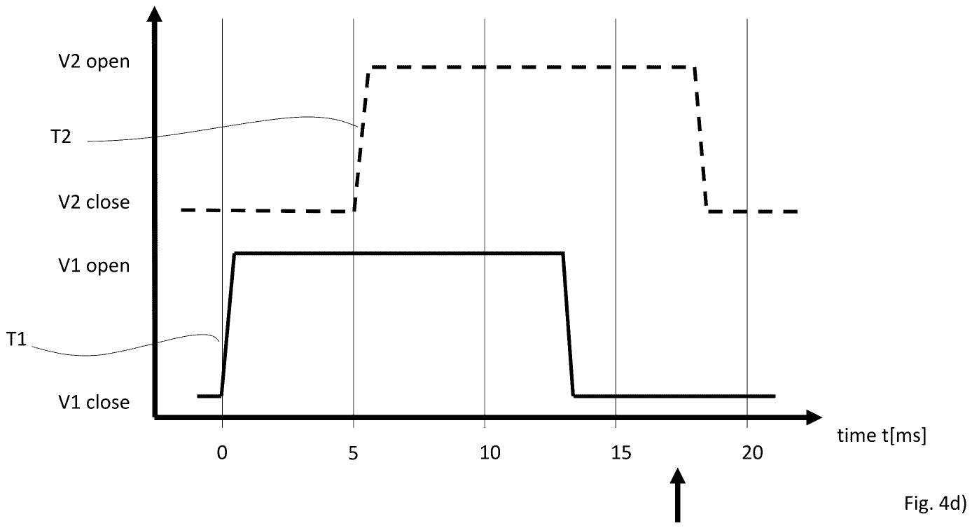

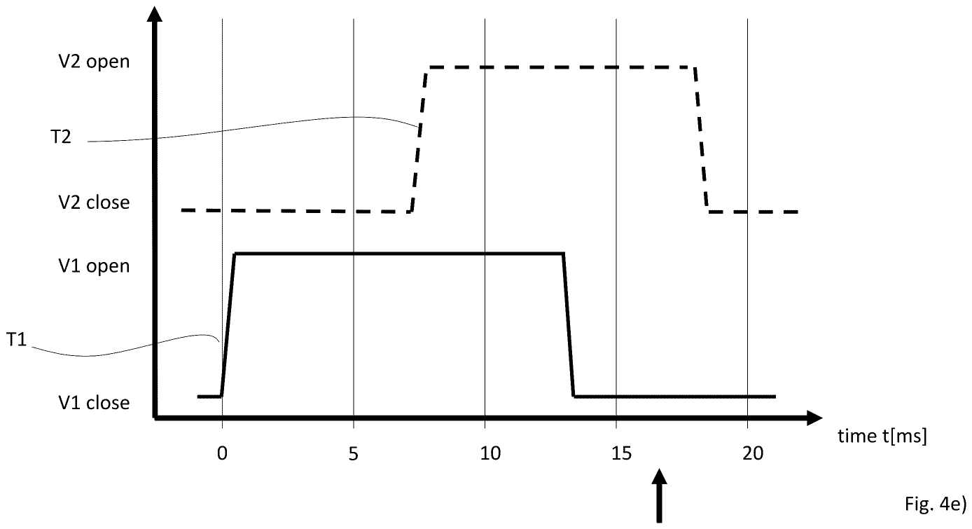

If, for example, as illustrated in the exemplary embodiment, a small number of discrete different overlap times are provided, which are given by a start of the second (first) activation time (with, for example, respectively constant end of the two activation times) offset differently from the start of the first (second) activation time, then, in the control state with the earliest start of the second (first) activation time (with, for example, constant supply pressure), a shorter actual acceleration of the projectile takes place in comparison with the other control states with later start of the second (first) activation time. The projectile is therefore accelerated to a comparatively lower speed and in this respect also requires more time until impact. With ever later start of the second activation time (or ever earlier end of the second activation time), the projectile speed therefore increases and, at the same time, the time of impact shifts forward relative to the start of the projectile movement. Preferably, in the first case, that is to say an overlap time at the end of a first activation time, the collision of the projectile with the applicator also remains within the overlap time in the control state with the highest projectile speed, that is to say the latest start of the second activation time. For illustration, reference is made to FIGS. 4 a) to e) and the description thereof.

Preferably, one of the two activation times can be of constant length in comparison between two control states with different overlap time (in this case including zero). In the case of an overlap time at the end of the first activation time, this preferably relates to this first activation time and, in the other case, correspondingly to the second activation time. This therefore means that, in these cases, the differences in the overlap time result from different lengths of the respectively overlapping other valve opening time or the temporal relationships between the two valve opening times. Preferably, the respectively other valve opening time is of variable duration between two such control states with different overlap time (wherein, for example, it could have the same end time in all or some of the control states, measured from the start of the earlier activation time).

It was already mentioned at the outset that apparatus of the type considered here typically carry out a plurality of acceleration, collision, and return transport processes of the projectile. In the prior art, the corresponding repetition frequency of such a periodic operation is regularly adjustable. Also in the present case, an iterative (not necessarily periodic) operation and a corresponding design of the apparatus are preferably considered, wherein the corresponding sequence does not necessarily have to begin with a movement in the forward direction.

For example, such a sequence can begin with a first pneumatic pressure pulse for movement of the projectile in the reverse direction into its position distal with respect to the applicator, in order to establish defined initial conditions. Principally, magnets at this distal end of the movement path of the projectile are already known in the prior art, with which the projectile is to be fixed. However, a projectile could be released from this fixing as a result of impacts and such a fixing could of course also be dispensed with.

Otherwise, the explanations do not necessarily relate to each individual movement process during such a movement sequence. The operating conditions can, as will be explained in more detail, also be changed during a sequence, with the result that the described overlap time possibly does not exist at all, for example in the case of a part of the movement sequences in the sequence.

According to a preferred configuration, the apparatus and in particular the control means can be adapted in such a way that no such overlap time exists in certain control states, but rather even a “separation time” (which is different from zero) is maintained between the end of the first (or second) activation time and the start of the second (or first) activation time.

For example, the first activation time can be ended significantly before the impact of the projectile onto the applicator and the second activation time can start, for example, directly after impact. Then, not the entire available time between the movement start of the projectile and the impact onto the applicator is used for the acceleration in the presence of accelerating pressure, but only a first part thereof. For example, the impact speed of the projectile could thus also be reduced without having to lower the accelerating pneumatic pressure. It may be desired, for example, to allow this to be at a higher value for a return which is as fast as possible (with the aid of the second activation time) than is momentarily required for the acceleration in the forward direction.

Moreover, the separation time can also lie completely or partially after the collision. Then, too fast a return movement or too high a speed at the end of the return movement can be prevented, for example, by a delayed start of the second activation time after the collision, without having to lower the accelerating pressure (for the forward direction). In this context, it should also be taken into account that the collision itself already brings about a certain acceleration of the projectile in the reverse direction according to the laws of momentum conservation.

Of course, both aspects can be combined, namely a part of the separation time before and another part of the separation time after the collision.

Of course, a further separation time can then also follow after the second activation time following the previously mentioned separation time (or also without such a separation time after the second activation time). For example, the second activation time does not have to last completely until the distalmost position of the projectile is reached.

Preferably, the first activation time is of variable length in at least two control states with different separation time, that is to say, for example, with a fixed portion of the separation time after the collision (including zero). Of course, the second activation time can also be of variable length, but, for example, in the case just described of a fixed portion of the separation time after the collision, can also be entirely constant, specifically preferably with regard to duration and/or start and end with respect to the collision as a reference.

In the simplest case, the pneumatic means can comprise a connection for supply from a pneumatic line network, for example in a hospital, or from a compressed gas cylinder. However, preference is given to a pneumatic compressor with which the apparatus according to the invention is locally independent and more mobile in comparison with a compressed gas cylinder. Pneumatic compressors are already known per se in conjunction with such apparatuses. However, the invention offers the particular aspect of not necessarily having to change the supply pressure in different control states with different impact speeds of the projectile. In other words, the compressor can run at the same rotational frequency in such different control states.

Of course, this can first of all simplify the controlling of the compressor, in particular if the latter principally runs at the same rotational frequency in the activated state. Furthermore, the compressor can be operated in the vicinity of its or at its maximum efficiency (with respect to the rotational frequency). Moreover, it is possible to match noise reduction measures, for example a damping mounting of the compressor or a noise-damping casing, to the vibration behavior of the compressor at the same rotational frequency.

A particular design possibility of the invention is based on being able to influence the impact physics between projectile and applicator directly and rapidly solely by changing valve opening times or valve opening time durations, specifically the impact speed and therefore the impulse upon impact. In comparison with a change in the supply pressure, this possibility of influencing is particularly rapid, such that, in an iterative operating state, in principle the impact speed/impact impulse of the combined forward and return movement can be changed from one impact process to the next. Such a rapid and free influencing or control action is not enabled by the prior art.

Typical impact speeds are in the range between 2 m/s and 30 m/s, but also in the case of conditions which change less rapidly or do not change. For impact physics, above all the impact pulse is important, which, in the case of typical projectile masses, can be between 1 g and 10 g, preferably between 2 g and 5 g, and therefore in a range from 2 μm/s to 300 μm/s. A range between 10 μm/s and 150 μm/s is preferred.

In a particular configuration, the apparatus has a measuring means, with which the passage of the projectile can be measured at a point of its movement path. This measuring means can be coupled to the control means. Thus, in such a form, for example, the passage of the projectile shortly before impact or quasi during impact onto the applicator can be detected, so that the activation times can be matched accordingly (in particular with regard to their start and their end) to the time of impact.

Such a detection can take place, for example, optically, for example, by a light barrier or the like, but preferably inductively using a measuring coil. This can detect the projectile by a residual magnetism of the projectile or purely inductively (by changing the leakage inductance).

The invention will be explained in more detail below on the basis of exemplary embodiments, wherein the individual features can also be essential to the invention in another combination within the scope of the claims.

In detail,

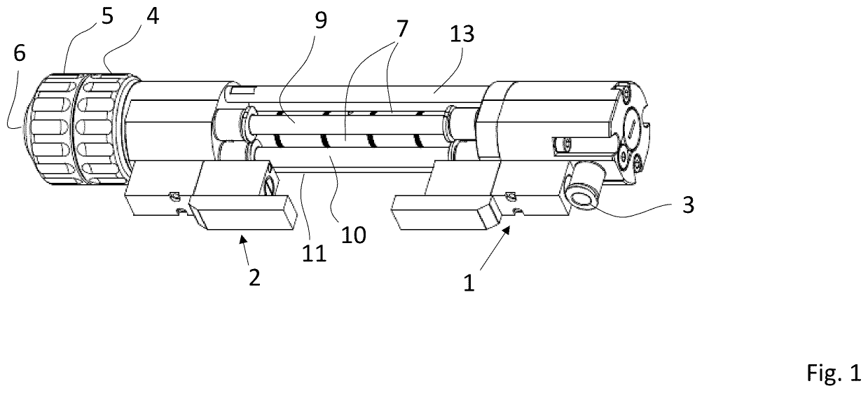

FIG. 1 shows a perspective illustration of an apparatus according to the invention, wherein a central housing part is omitted for the sake of clarity;

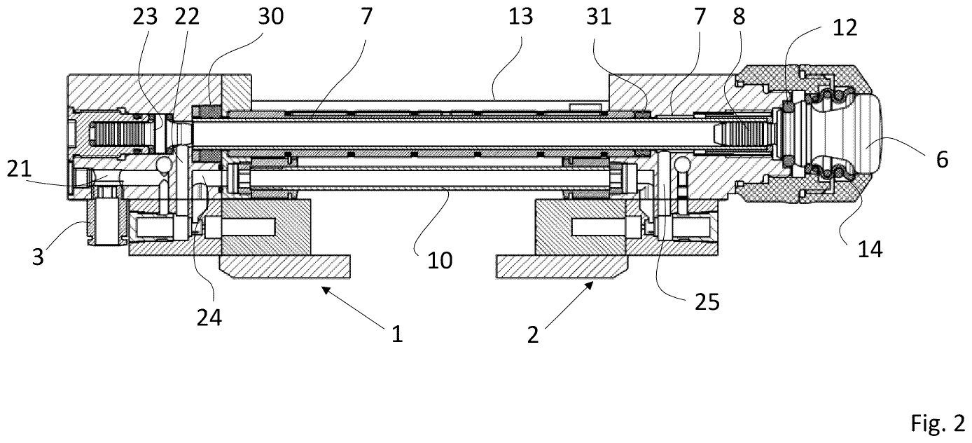

FIG. 2 shows a longitudinal section through the apparatus from FIG. 1 in the right-left-reversed position with respect to FIG. 1;

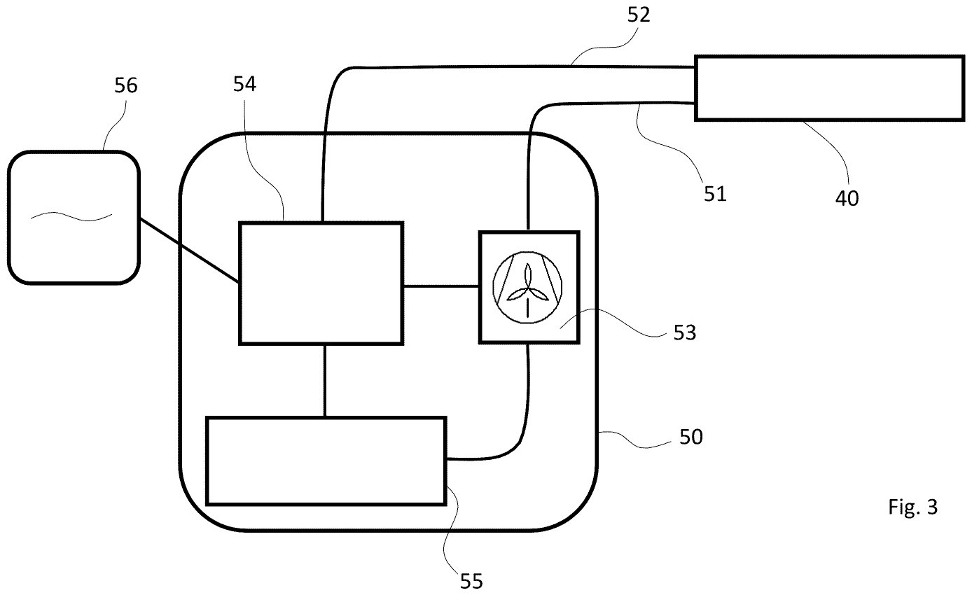

FIG. 3 shows a schematic diagram of the handpiece with an associated basic apparatus;

FIG. 4 shows a sequence of schematic time diagrams 4 a) to e) for explaining the mode of operation;

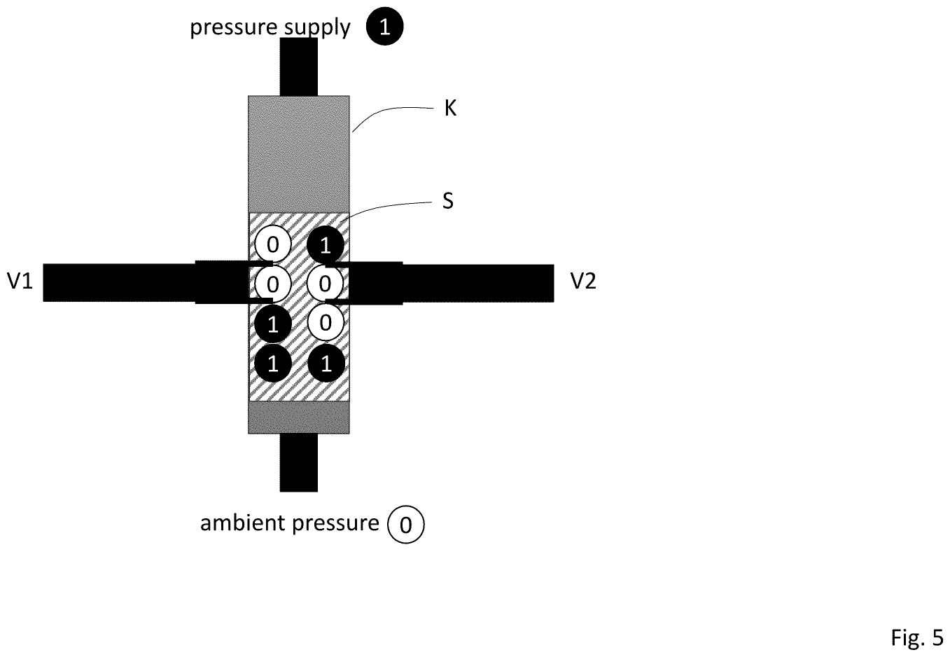

FIG. 5 shows a schematic illustration of a combination valve for explaining an alternative exemplary embodiment to FIGS. 1 and 2;

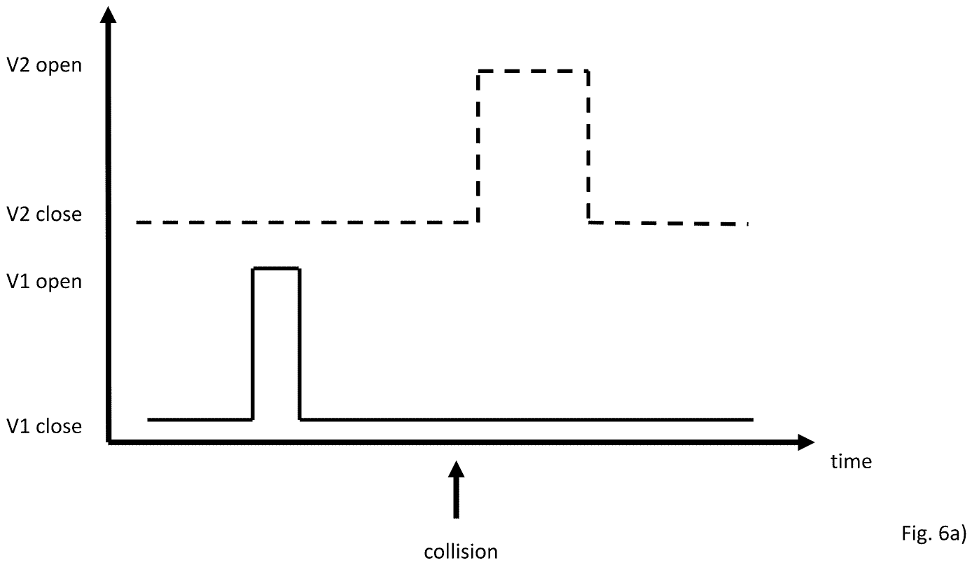

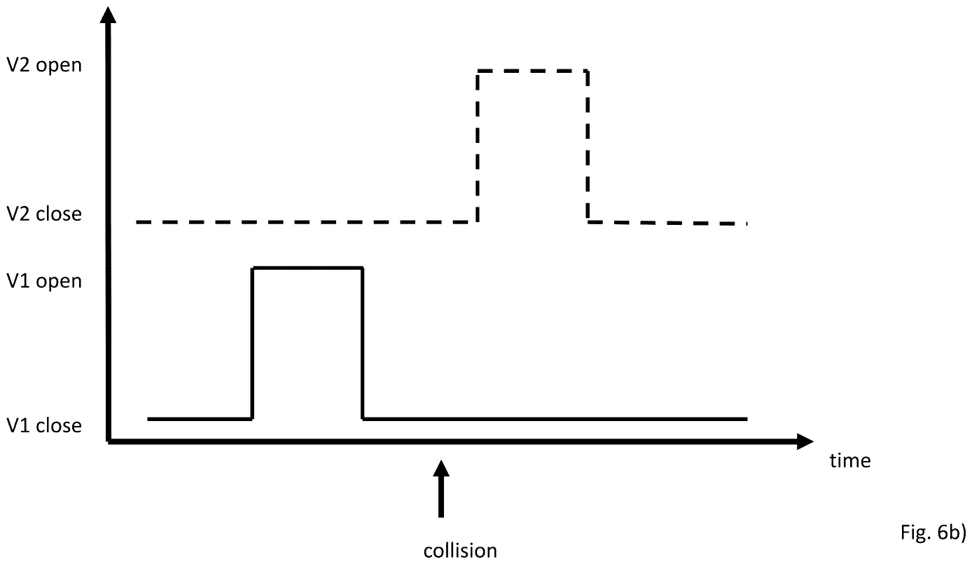

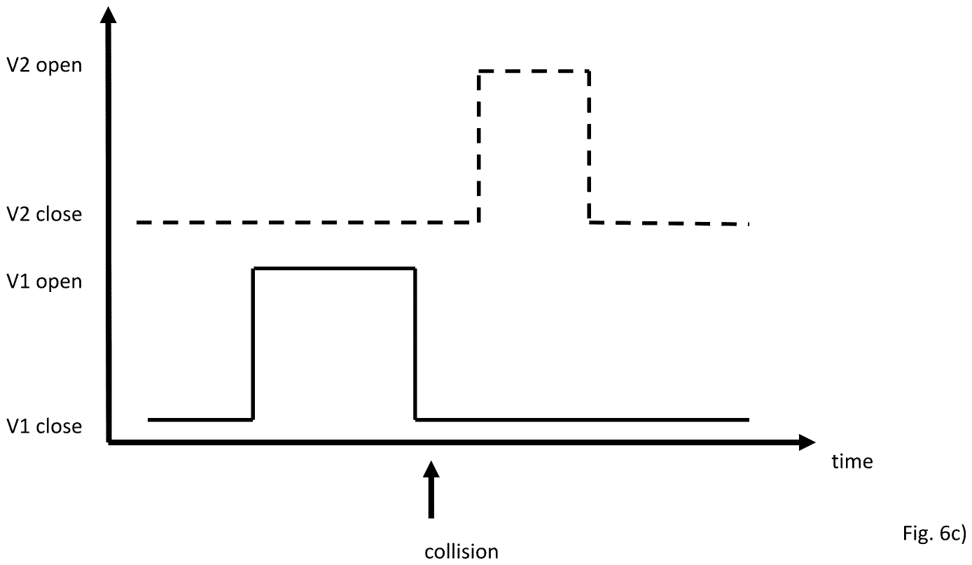

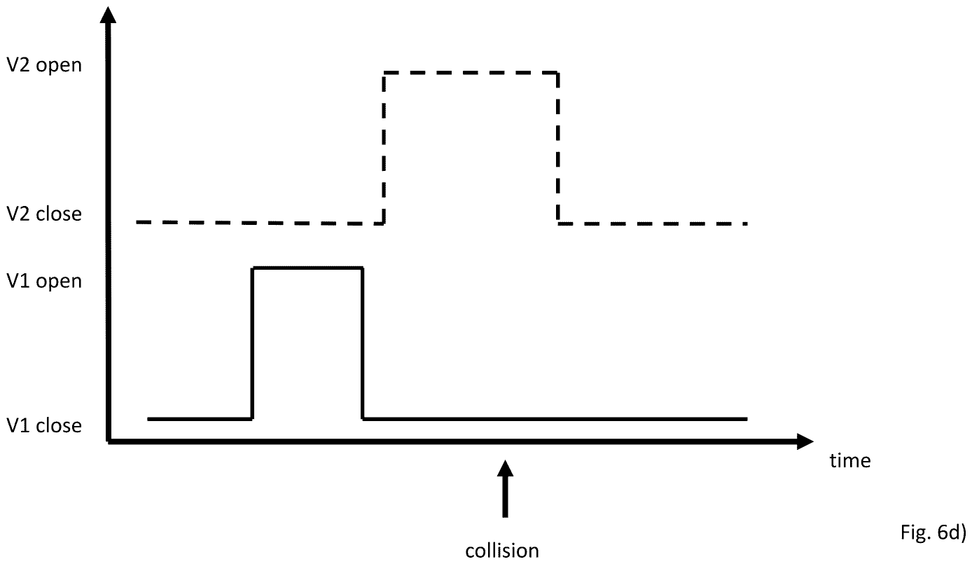

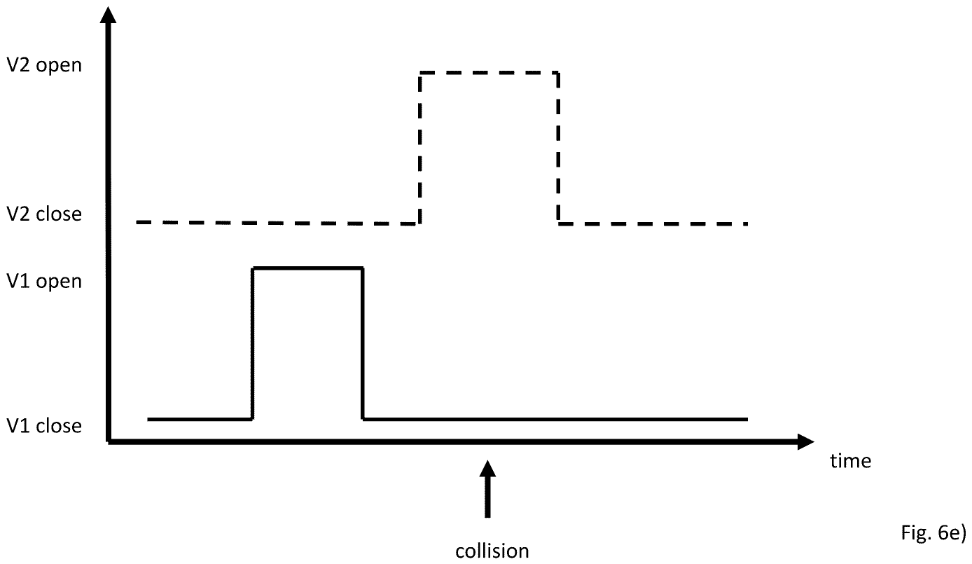

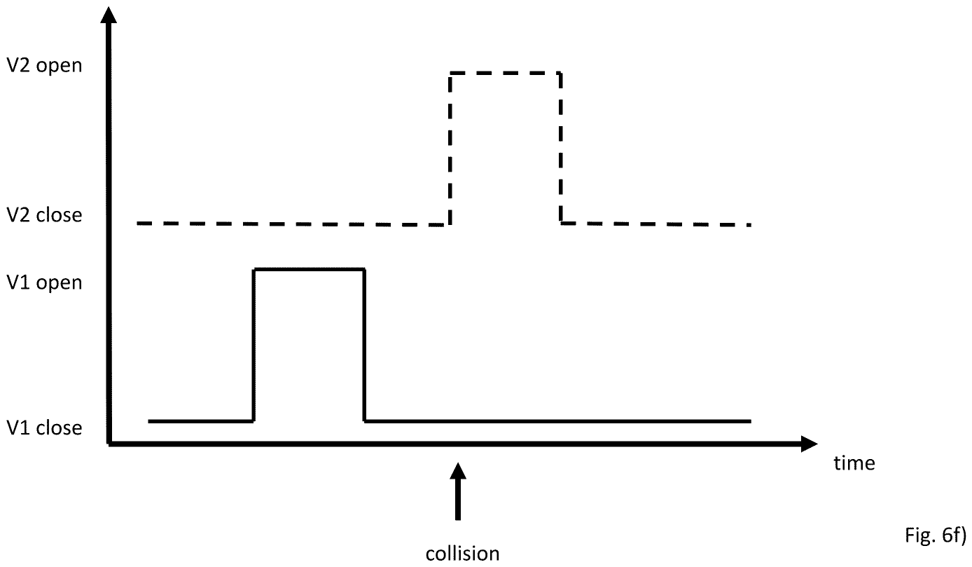

FIG. 6 shows a sequence of schematic time diagrams 6 a) to f) for explaining further control states in addition to FIG. 4;

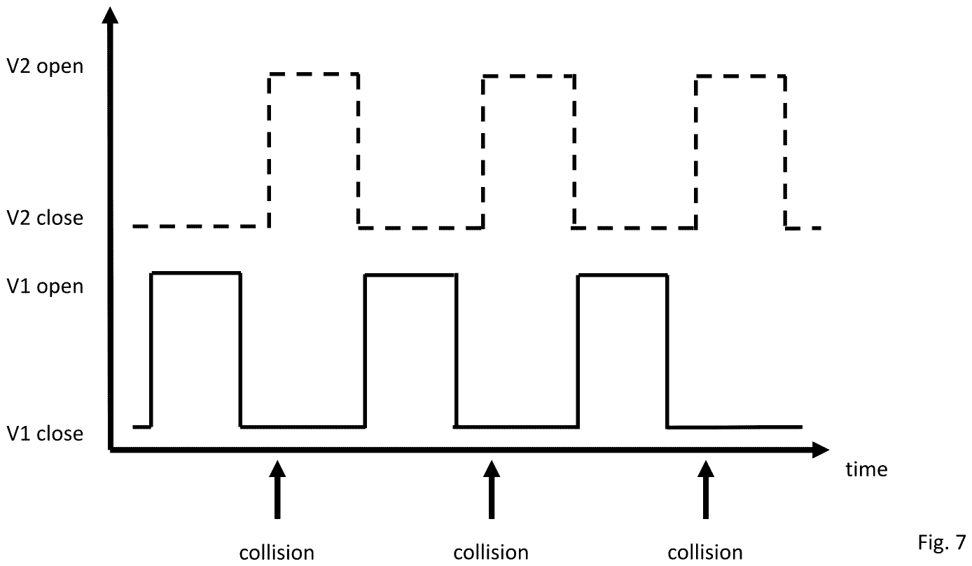

FIG. 7 shows a further schematic flow diagram for explaining the periodic mode of operation;

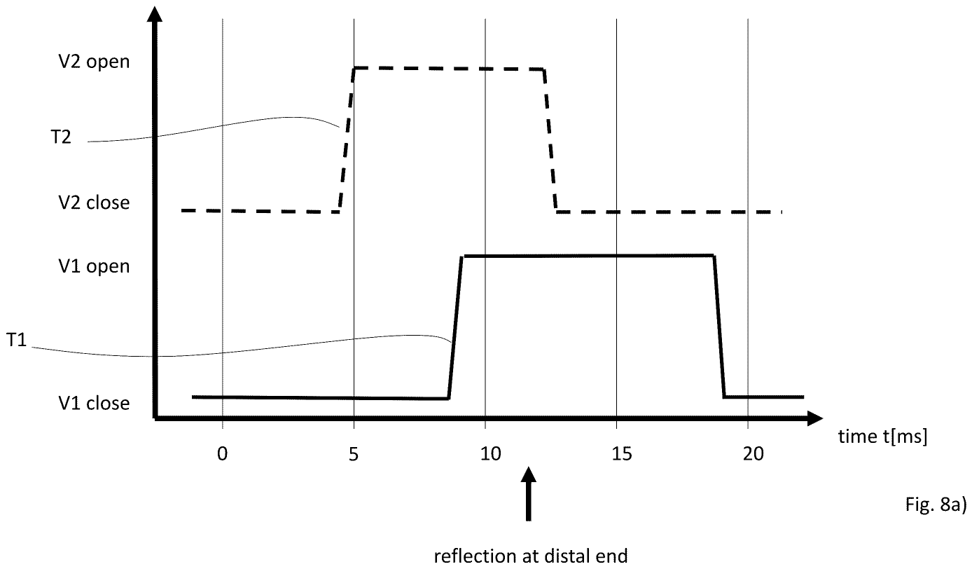

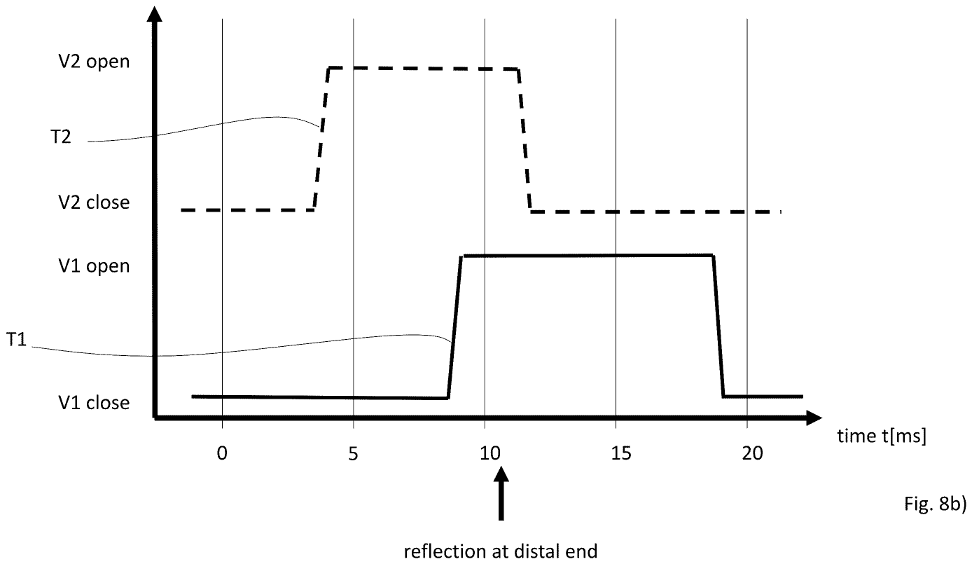

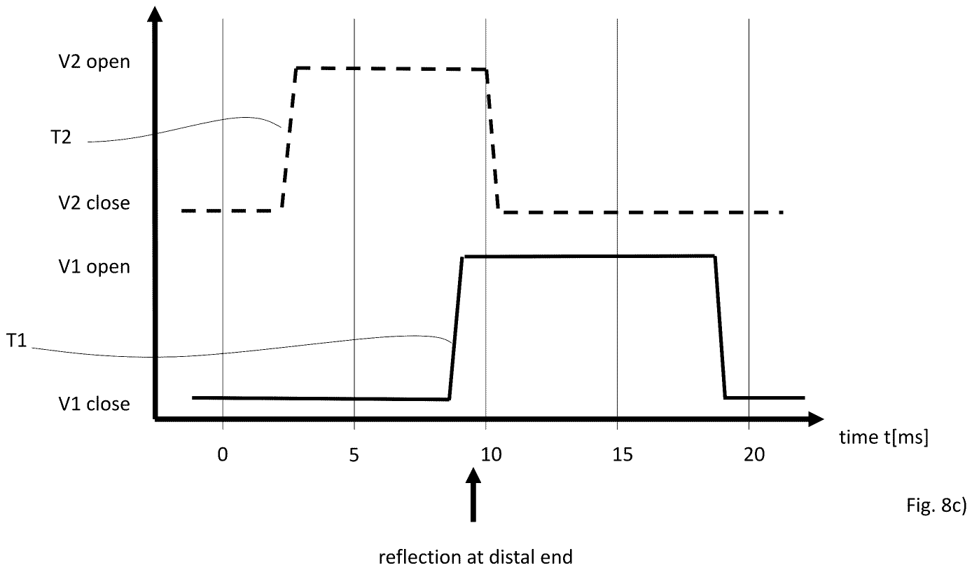

FIG. 8 shows a sequence of schematic time diagrams 8 a) to c) for further explanation of the mode of operation;

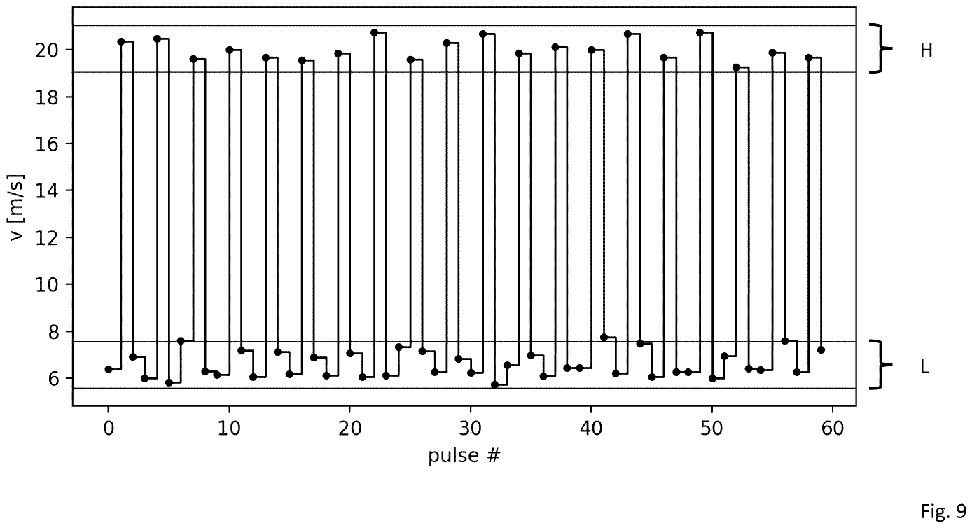

FIG. 9 shows a recurring sequence of two different projectile speed levels in direct succession, two pulses at low speed following directly after a pulse at high speed;

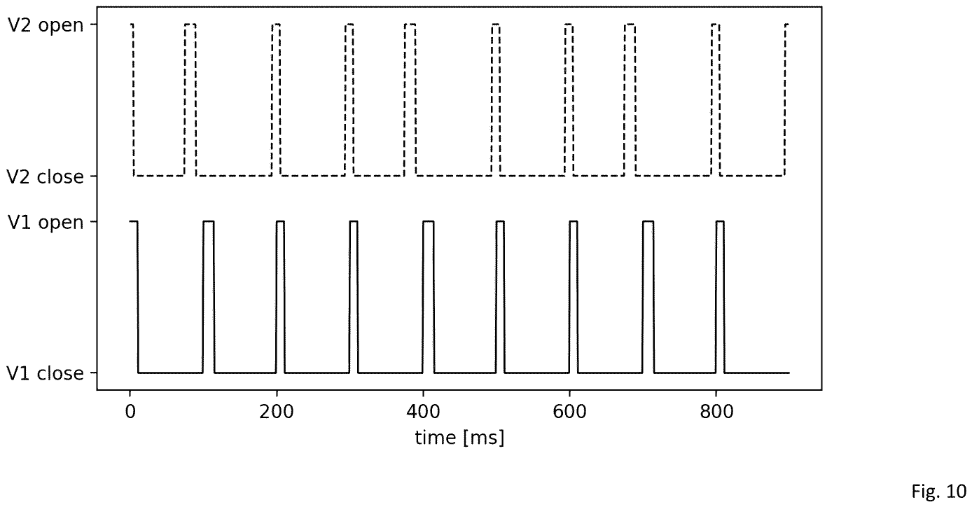

FIG. 10 shows a control sequence for controlling the valves V1 and V2 according to the sequence of two different projectile speed levels shown in FIG. 9;

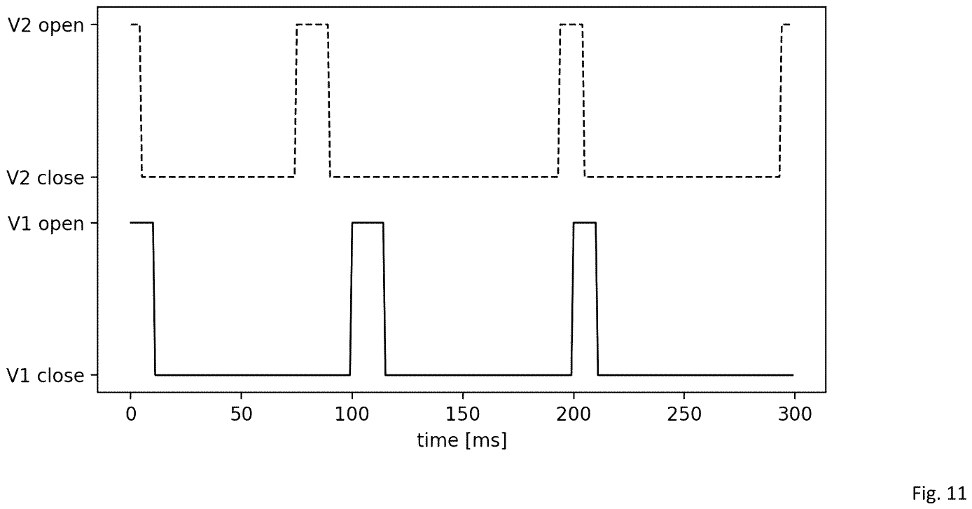

FIG. 11 shows a higher temporal detailing of the first 300 ms from the control sequence of FIG. 10;

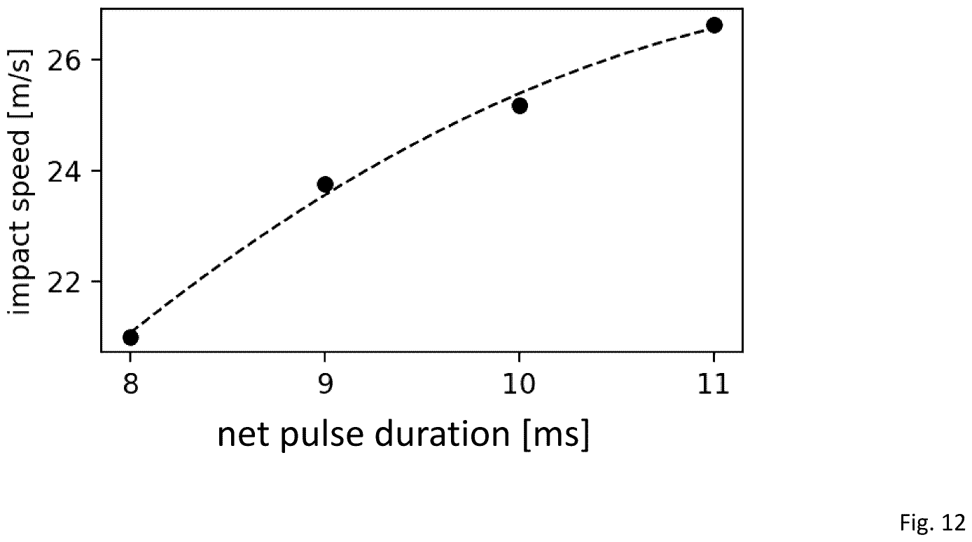

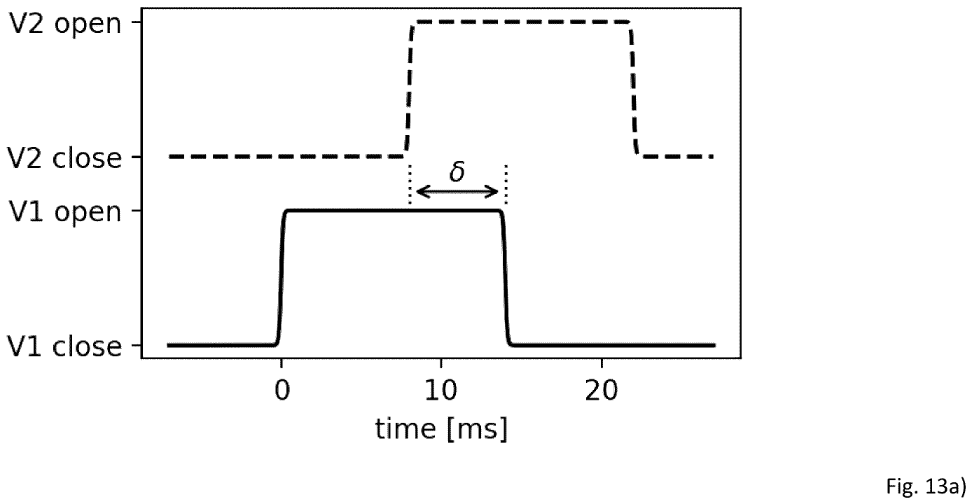

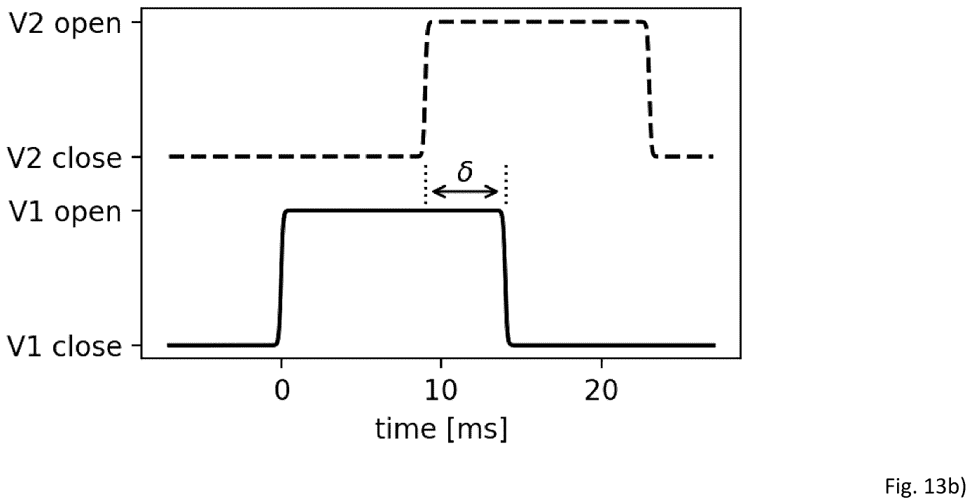

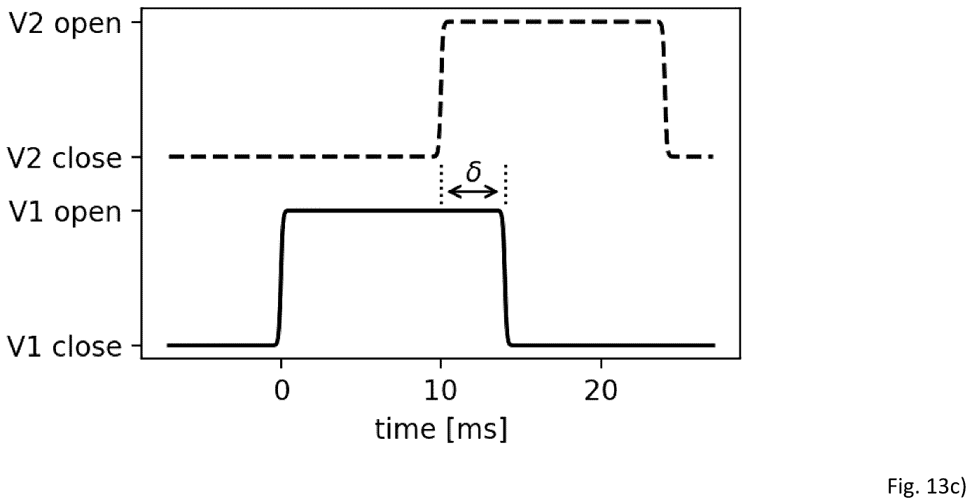

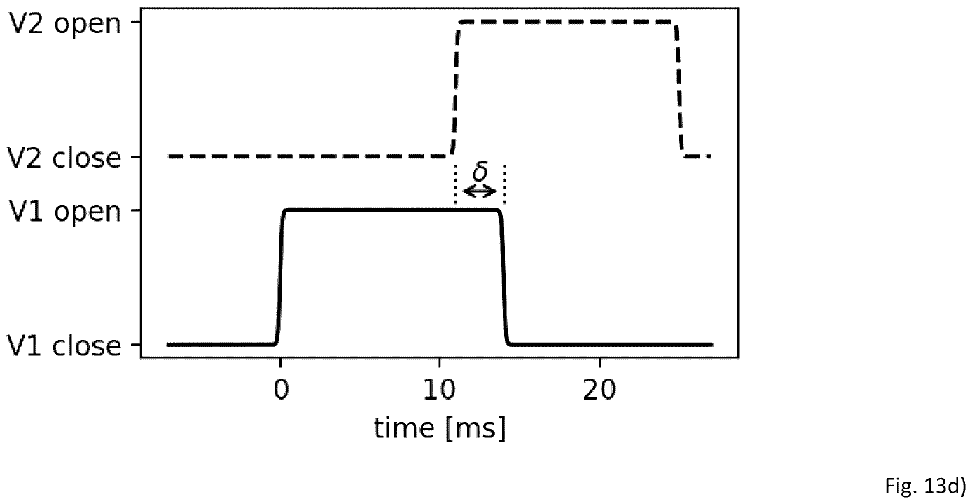

FIG. 12 shows a relationship, determined by actual measurements, between the portion of the first valve opening time before the overlap time on the horizontal axis and the projectile collision speed on the vertical axis, specifically with respect to the representations in FIG. 13;

FIG. 13 shows a sequence of time course representations 13 a) to d) for illustration of the actual valve opening times and the respective overlap time therebetween with respect to the individual measurement points in FIG. 12.

FIG. 1 shows a handpiece of an apparatus according to the invention in a perspective view with pneumatic valves pointing to the front-left, namely a first valve 1 and a second valve 2. A pneumatic supply connection 3 can be seen on the right and two screw rings 4 and 5, which are respectively corrugated on the outside for easier handling, for holding the applicator 6, which will be explained in more detail below, can be seen on the left. The latter can still be seen on the far left in FIG. 1 with its patient-facing surface and is otherwise shown in FIG. 2. It could also be constructed in multiple parts.

A number of tubes running in the transverse direction can be seen in the central region of the apparatus from FIG. 1, wherein the central tube with the reference numeral 7 contains and guides the projectile 8, which can be seen in section in FIG. 2. Two parallel pneumatic connecting pipelines 9 and 10 can be seen in front of this between the two valves 1 and 2, wherein the pipeline 9 serves for supplying a pressurization/pressure application to the second valve 2 and the pipeline 10 conversely serves for ventilation of this second valve 2 via an outlet provided in the first valve 1. In this exemplary embodiment, this number of pipes is surrounded by a housing cover 11, which is shown in FIG. 1 by the line below the pipeline 10 and the two lines above the projectile guide pipe 7. This housing cover 11 runs in the rear region in FIG. 1 and comprises only a part of the circumference. At its respective axial edges, it is designed in a manner similar to a flanging by means of a rounded turn-over inwardly in a manner favorable to the grip, which is indicated in FIG. 1 at the upper edge. The housing cover 11 can thus serve as a handle during practical handling. The spacer 13 stabilizes the construction and connects the two ends of the handpiece mechanically.

A flexible compressed air feed line (cf. 51 in FIG. 3) leading from a pneumatic compressor to the apparatus is not shown here and is to be connected to the already mentioned connection 3. Analogously, an electronic control line (52 in FIG. 3) from an external controller to the valves 1 and 2 is not shown, which can be designed in a uniform manner with the compressed air feed line.

FIG. 2 shows a longitudinal section along an imaginary central longitudinal axis of the already mentioned cylindrical shape of the overall apparatus, which is at the same time a central longitudinal axis of the projectile guide tube 7. For illustration of the dimensions: in this exemplary embodiment, the length of the projectile guide tube 7 is 145.5 mm and the remaining illustration in FIG. 2 is to scale. In this projectile guide tube, the projectile 8 is shown on the right in FIG. 2 and thus abuts the applicator 6, which is held by the described screw ring 4 and 5 in a manner known per se. In this case, the applicator 6 is elastically mounted in the axial direction by a bellows-like elastomer ring 14 and is pneumatically sealed by a further elastomer ring 12. Alternatively, an apparatus design with regard to the applicator 6 and its holding and sealing according to, for example, EP 2 529 679 (also independently of the cap shown there) or EP 2 095 843 (also independently of the ceramic material discussed there) is also possible and preferred.

FIG. 2 shows on the left an inner channel 21, which connects the pneumatic connection 3 to the first valve 1. The first valve 1 can accordingly switch a supply pressure applied to the pneumatic connection 3, depending on the control, to a radial channel 22, which opens under a damper elemenT23 and is thus connected to the inner volume of the projectile guide tube 7. Via this channel 22, the projectile is therefore acted upon or accelerated during a first activation time in the direction of the applicator 6. Independently of this, the pneumatic supply pressure is passed on to the second valve 2 via the channel 24 and the pipe 10.

In the second alternative switching position, the channel 22 and thus also the inner volume of the projectile guide tube 7 between the distal end (on the left in FIG. 2) and the projectile 8 are ventilated.

In the second valve 2, which is constructed principally mirror-symmetrically with respect to the first valve 1, the pneumatic supply pressure applied via the pipe 10 can alternatively be passed radially upward via the channel 25 to a volume surrounding the projectile guide tube 7 (to be seen in FIG. 2 as a slot above and below the pipe 7), which leads from the connection of the channel 25 to the right, i.e., in the direction of the applicator 6, and is connected there between the applicator 6 and the end of the projectile guide tube 7 proximal to it to the inner volume of the projectile guide tube 7 (apart from the presence of the projectile 8 shown there in FIG. 2). Via the channel 25, the pneumatic supply pressure can therefore be applied switchably to the inner volume of the projectile guide tube 7 between the applicator 6 and the projectile 8. In this example, however, the pneumatic connection is somewhat poorer as a result of a smaller effective opening cross section than on the opposite side of the projectile guide tube 7, so that, here, at higher air flow speeds (higher frequencies, higher pressures), delays become noticeable earlier or more strongly.

Alternatively, in the other switching position, the second valve 2 can block the connection of the inner volume of the pipe 10 to it and ventilate the channel 25 and thus the inner volume of the projectile guide tube 7 on the right of the projectile 8, i.e., connect it to the external atmosphere via a pneumatically highly conductive connection.

The two valves 1 and 2 can therefore apply pneumatic pressure to the projectile from both sides, namely independently of one another and thus simultaneously or alternately, or can ventilate the interior of the projectile guide tube 7 on both sides.

The reference numeral 30 in FIG. 2 denotes a ring-shaped permanent magnet at the end, which is distal with respect to the applicator 6, of the movement path of the projectile 8 (coinciding with the length of the projectile guide tube 7). With this magnet 30, the projectile 8 constructed from ferromagnetic material can be easily fixed at this distal end of the movement path. By unilateral pressurization by means of the valve 2, the projectile can furthermore be returned to this position and optionally also additionally held there, in particular at the start of operation or in the case of a non-ferromagnetic projectile. In this respect, the permanent magnet 30 can optionally also be omitted, especially when the reflections, which are still to be explained in the further course, at this distal end of the movement path are to be made possible there even at low impact speeds of the projectile 8.

Reference 31 denotes a point at which the passage of the projectile 8 through the corresponding point of the movement path could be detected with a measuring coil, this point lying relatively close to the applicator 6. In the simplest case, a slight residual magnetism of the projectile 8 is used here, but the changing of the inductance of the coil 31 could of course also be detected and evaluated using alternating current technology. The collision of the projectile 8 with the applicator 6 can also be determined by the use of a microphone or movement sensor in the experimental setup. In addition, the impact speed of the projectile 8 can be determined in the experimental setup, for example, with two light barriers positioned just in front of the applicator 6.

FIG. 3 shows a block diagram with the apparatus shown in FIGS. 1 and 2 at the top right, to be precise denoted in summary by the reference numeral 40. This apparatus 40 is a mobile handpiece to be held in the hand, as is already known per se from relevant apparatuses from the prior art. It is connected via two lines 51 and 52 to a base station 50, which contains a pneumatic compressor 53 and a controller 54. The compressor 53 is connected via the line 51, namely a pneumatic flexible hose line, to the handheld apparatus 40 and the controller 54 is connected via the electrical line 52 (optionally integrated with the line 51), via which the controller can access the already mentioned two valves 1 and 2 and supply them with power. In addition, communication with the handpiece 40 can take place via the line 52, in particular if a controller or a part of the controller is additionally provided there.

Moreover, the controller 54 also controls the compressor 53 with respect to its rotational frequency and, of course, the switching on and off and, in turn, is supplied with power by a mains apparatus 55, just like the compressor 53. However, a pressure control influencing the rotational frequency or a control valve can also be integrated in the compressor 53. In addition, the controller 54 is connected to a display 56, which can be installed in the basic apparatus 50 or can also be implemented separately therefrom. The basic apparatus 50 is operated via a touch-sensitive display 56 and/or via an arrangement of buttons, not shown here.

The user can thus control the function of the apparatus 40 on the basis of such buttons and in any case on the basis of the display 56, wherein the controller 54 specifies in particular the opening and closing times and thus also the opening durations of the two valves 1 and 2. Partial tasks of the controller 54 can also be integrated in the handpiece 40, particularly with respect to the controlling of the valves 1 and 2.

For a basic understanding of the controlling of the two valves, reference can be made to the earlier patent EP 2 213 273 B1. With regard to the dimensioning in particular of the projectile guide tube and of the projectile, the exemplary embodiment therein corresponds largely to the above explanations and to FIGS. 1 and 2 with the exception of the existence of the second valve 2 and the omission of the counterpressure chamber. In addition, in the exemplary embodiment cited, a specific valve opening time of the single valve there is assumed at a specific pressure, whereas the projectile acceleration in the present case takes place variably by means of the portion of the first valve opening time also outside the overlap time and therefore also at a constant pressure. For the following explanations, a pressure of 4 bar can be assumed by way of example. This results in the following exemplary table of values:

Table Of Values

| | | | | | |
|---|---|---|---|---|---|
| projectile speed [m/s] | 10 | 12 | 14 | 16 | 18 |
| opening time valve 1 [ms] | 0 | 0 | 0 | 0 | 0 |
| closing time valve 1 [ms] | 13.0 | 13.0 | 13.0 | 13.0 | 13.0 |
| opening time valve 2 [ms] | 2.6 | 3.0 | 3.7 | 5.0 | 7.1 |
| closing time valve 2 [ms] | 18 | 18 | 18 | 18 | 18 |
| on-duration valve 2 [ms] | 15.4 | 15.0 | 14.3 | 13.0 | 10.9 |
| overlap time [ms] | 10.4 | 10.0 | 9.3 | 8.0 | 5.9 |

FIG. 4 shows a sequence of five individual schematic time diagrams 4 a) to 4 e) corresponding to the above table, in which the opening and closing process of the first valve 1 is respectively denoted by the curve denoted by T1 and the opening and closing process of the second valve T2 is analogously denoted by the curve denoted by T2. The increased curve part thus corresponds respectively to the first/second activation time.

In comparison, it can be seen that the first activation time in all five control states on the (arbitrary) time axis in the horizontal direction begins at 0 ms and ends aT13 ms. In contrast, the second activation time with regard to its beginning shifts from initially 2.6 ms in FIG. 4 a) stepwise to 7.1 ms in FIG. 4 e), whereas the second activation time in all five illustrations ends aT18 ms. Accordingly, there is an overlap time in all control states, namely from approximately 3 ms to 13 ms in FIG. 4 a) to still from approximately 7 ms to 13 ms in FIG. 4 e), wherein this overlap time decreases stepwise, namely corresponding to the increasingly delayed beginning of the second activation time. In this respect, the pneumatic application by the second valve 2 with regard to the return of the projectile 8 is active in all five control states.

In the cases illustrated in FIG. 4, impact speeds of the projectile 8 onto the applicator 6 of (in this order from a) to e)) 10 m/s, 12 m/s, 14 m/s, 16 m/s, and 18 m/s are realized. This corresponds to impulses of 30 μm/s to 54 μm/s with a projectile mass of 3 g. The opening time of the valve 1 is constanT13.0 ms. The closing time of the second valve also remains constant aT18 ms.

More precisely, FIGS. 4 a) to e) show the electrical control times of the two valves 1 and 2, that is to say the output signals of the controller 54. The valves 1 and 2 are spring-assisted solenoid valves which open purely magnetically and close by the force of the spring which is tensioned in the process when the magnet is no longer loaded. The movements of the valve body are accordingly somewhat delayed with respect to the control signals illustrated, specifically by an estimated 4 ms during opening and 2 ms during closing. The overlap times are therefore actually approximately 2 ms shorter than illustrated.

In the case of a so-called pilot valve with pneumatic assistance during opening, the situation would be qualitatively comparable.

In FIG. 4 a) (of course at a start of the projectile movement at the left-hand end of the movement path in FIG. 2 at 0 ms), the collision with the applicator takes place after the overlap time and also after the end of the second activation time, that is to say at approximately 18 ms to 20 ms, wherein this collision time shifts ever further to the left in the following figures and, starting from FIG. 4 c), lies within the second activation time. The projectile speeds measured (optically in an experimental setup) are between 10 m/s in FIGS. 4 a) and 18 m/s in FIG. 4 e) and are therefore in a ratio of 1:1.8. In this case, it can be imagined in a simplified manner that the projectile is accelerated linearly over time before the second activation time and is then moved further at approximately the speed achieved (disregarding pneumatic flow effects and projectile friction); in fact, the projectile speed will probably increase somewhat less than linearly over time and will slightly decrease in an approximately force-free state during the overlap time on account of friction. After the overlap time, the projectile 8 is braked in all individual illustrations by the still present pneumatic application of pressure by the second valve, wherein, in the cases 4 a) and 4 b), after the end of the second activation time, the projectile again covers a short distance until the collision in an approximately force-free manner in the above sense.

In particular in FIG. 4 a), it is noticeable that the remainder of the second activation time after the overlap time is significantly longer than the (initial) remainder of the first activation time before the overlap time. This may be surprising because the same supply pressure abuts both valves and, according to the real values from the above table of values, a collision nevertheless takes place in FIG. 4 a) aT10 m/s. One reason may be that the second valve 2 according to FIG. 2 is pneumatically connected significantly more inefficiently to the interior of this pipe 7 in the region of the right-hand end of the projectile guide tube 7 than the first valve 1 at the left-hand end. This has to do with the fact that, at the right-hand end, as can be seen in FIG. 2, the projectile 8 is prevented from flying out to the right by a cross-sectional constriction (catching device). This has safety reasons if an apparatus without a mounted applicator were inadvertently put into operation. In this respect, the corresponding pipe end apparently fills more slowly during the opening of the second valve 2 and, therefore, there is a greater delay between the valve switching processes and the actual exertion of force on account of the pneumatic application of pressure by the second valve in a dynamic view.

Otherwise, the figures show that a portion of the second activation time after the collision lies only in the illustrations 4 d) and 4 e). This does not disturb any further, because the projectile is pushed back in the sense of momentum conservation by the collision itself in the sense of the impact between a typically lower-mass projectile and a higher-mass applicator. The remainder of the second activation time after the end of the first activation time in FIGS. 4 d) and e) in this respect only additionally ensures the return movement into the starting position.

Of course, the control times could be adapted to the extent that the overlap time ends approximately respectively at the collision time. In particular, this could be done with a temporal determination of the collision time by the possibility, already illustrated on the basis of FIG. 2, of a measuring coil 31 in the vicinity of the applicator 6. If the collision time is intended to lie relatively precisely at the end of the overlap time (or at another fixed point), the control time scheme would be somewhat more complicated, because the first activation time would have to be ended differently early (from FIG. 4 a) to FIG. 4 e) always earlier). However, the speed of the projectile movement, in particular of the return movement, could be increased. In this case, it could also be of interest in the case of the higher projectile speeds to provide the end of the second activation time differently and in the case of an increasing projectile speed earlier, in order to achieve an even higher repetition frequency range.

Of course, in the case of another exemplary embodiment with a “combination valve”, very similar relationships can be generated as illustrated in FIG. 4 in the diagrams a) to e), in which case, however, the overlap time would then mean a different switching state of the valve. Such a combination valve is illustrated schematically in FIG. 5. In this case, the letter K denotes the combination valve, which accordingly replaces the two valves 1 and 2 from FIGS. 1 and 2. Two lines V1 and V2 are illustrated on the right and left, of which V1 means a connection to the left-hand side (according to FIG. 2) of the projectile guide tube 7, for example via the channel piece 22 (analogously to the first valve 1). Accordingly, the right-hand line V2 means a connection to the right-hand side of the projectile guide tube 7 (analogously to the second valve 2), i.e., for example via the channel piece 25.

The upper line is denoted in FIG. 5 by the keyword “pressure supply” and the symbol “1” (not to be confused with the reference numeral 1) for the first valve; analogously, the lower line connection is denoted by the keyword “ambient pressure” and the figure-internal symbol “0”, i.e., means a ventilation opening.

There is a slide S, illustrated symbolically, in the combination valve K, which slide can be displaced in the vertical direction (with respect to FIG. 5) between four different switching positions. In the uppermost position, as illustrated in FIG. 5, the connection V1 is ventilated and the connection V2 is applied with the pneumatic supply pressure, in the third position, from above, vice versa, and in the second position, which has just been switched actively, from above, both connections V1 and V2 are ventilated. Finally, the lowermost position shows a simultaneous pressurization of both connections V1 and V2.

It would therefore be possible to imagine a combination valve K constructed in this or a similar manner instead of the two individual valves 1 and 2 from the exemplary embodiment in FIGS. 1 and 2, wherein the remaining explanations and in particular FIGS. 3 and 4 also apply analogously thereto.

Owing to the possibility of controlling the impact speed of the projectile 8 solely via the switching operation of the two valves 1 and 2, the pneumatic compressor 53 (FIG. 3) runs at a predefined fixed operating frequency at which it has a maximum efficiency. In addition, the pneumatic compressor can be particularly effectively damped in terms of vibration and noise at a predefined operating frequency.

Principally, the control means 54 can vary the impact speed and also the time interval between the collisions between the projectile 8 and the applicator 6 from one to the next individual operation. It can therefore influence the impact physics significantly more rapidly and more variably and is in particular not tied to periodic operations.

FIG. 6 shows schematic time diagrams similar to FIG. 4 in the individual illustrations a) to c), but with a separation time between the controlling of the valve 1 from FIGS. 1 and 2, which is represented by the solid line at the bottom, and the controlling of the valve 2, which is illustrated by the dashed line at the top. In FIG. 6 a), there is a relatively short activation pulse for the valve 1, whereby the projectile is accelerated and then “flies on” for a substantial part of the movement path without further pneumatic application after the end of this first activation time. In contrast to the overlap times illustrated in FIG. 4, however, both sides of the tube interior are ventilated (and not pressurized) in this movement phase.

After a certain time, a collision with the applicator, which is shown symbolically in FIG. 6 a), occurs and, relatively shortly thereafter (in addition to the already indicated return movement of the projectile solely on account of this collision), a returning pneumatic pulse occurs as a result of the second activation time according to the dashed line in FIG. 6 a). The projectile is thus moved back into the starting position again and is available for a new cycle.

In the individual illustrations b) and c), the explanation applies precisely in principle in the same way, wherein the first activation time is lengthened in a stepwise manner and the separation time between the first and the second activation time is thus shortened in a stepwise manner. The collision time consequently moves somewhat to the left, which is illustrated symbolically. Accordingly, the projectile strikes the applicator at an ever higher speed.

In all three diagrams a) to c), the activation time of the second valve lies after the collision. In the first two control states in FIGS. 6 a) and b), the larger part of the separation time lies before the collision, following it in the third case c).

In FIGS. 6 d) to f), in contrast to FIGS. 6 a) to c), the length of the first activation time is left unchanged (and corresponds to FIG. 6 b)). In contrast to the first three illustrations, however, a part of the second activation time lies before the collision, specifically the predominant part in case d), approximately half in case e) and only a very small portion in case f). The illustration FIG. 6 b), in which the second activation time then lies completely after the collision, can be thought of to some extent as a continuation, but this is not particularly important now.

These illustrations illustrate a further possibility of controlling the speed of the projectile during the collision. In FIG. 6 d), the projectile is namely pneumatically accelerated over the first activation time in a manner similar to that in FIG. 6 b), but then, in contrast to case b), flies only relatively shortly without force, in order then to be delayed by an opposing pneumatic pressure as a result of the beginning of the second activation time (dashed above). Since in case d) the delay time corresponds approximately to the acceleration time and the same pressure level can be assumed, the projectile strikes the applicator at a minimum speed and is then moved back again by the remainder of the second activation time.

In cases e) and f), the separation time between the two activation times is longer and therefore the portion of the second activation time before the collision is smaller in steps, which leads to an increasing projectile speed during the collision despite an unchanged first activation time.

In this respect, it is necessary to imagine a controller (according to FIG. 3) which can set control states according to the partial illustrations in FIG. 4 and further control states according to the partial illustrations just explained in FIG. 6. In both cases, the projectile speed during the collision can be influenced by valve switching times with a constant pressure.

FIG. 7 shows approximately a sequence of three operations corresponding to FIG. 6 e). In this case, as a result of the second activation times shown in dashed lines, the projectile is respectively brought back into the starting position again in order then to be accelerated from the chronologically following first activation time again in the direction of the applicator. This figure is intended merely to illustrate the possible periodicity of control states, which of course also applies in an analogous manner to the other partial illustrations in FIGS. 4 and 6. In addition, it can be imagined that the successive processes can have deviations from one another, such that the impact process can thus be changed rapidly and freely from one repetition operation to the next.

FIG. 8 shows a sequence of three individual schematic time diagrams 8 a) to c) in which the opening and closing process of the first valve 1 is respectively illustrated by the curve denoted by T1 and the opening and closing process of the second valve T2 is analogously illustrated by the curve denoted by T2. The increased curve part thus corresponds respectively to the first and second activation time. In comparison with the time diagrams from FIG. 4, here the second valve is opened corresponding to the curve T2 chronologically before the first valve corresponding to the curve T1. By varying the overlap between the two activation times, the reflection at the distal end of the movement path takes place earlier or later, as indicated on the horizontal axis in the three figures. In this example, both activation times are respectively of identical length per se (in comparison of the three individual illustrations with one another). However, the second activation time shifts further forward from FIG. 8 a) to FIG. 8 b) and then to FIG. 8 c) relative to the first activation time, with the result that the overlap time decreases. Because the portion of the second valve opening time (before the overlap time) which is effective for the reverse acceleration is greater in FIG. 8 c) than in FIG. 8 b) and is again greater there than in FIG. 8 a), the projectile speed present during the reflection at the distal end is correspondingly greater. Therefore, the projectile also moves again in the direction of the applicator at a correspondingly higher speed after the reflection at the distal end. In addition, the portion of the first valve opening time (after the overlap time) which is effective for the corresponding additional acceleration is also greater, as shown by the comparison of FIGS. 8 a) to c), with the result that the collision speed during the collision with the applicator increases from FIG. 8 a) to FIG. 8 b) and finally to FIG. 8 c) for two reasons.

FIG. 9 shows a recurring sequence of pulses with two different projectile speed ranges (during impact), which are denoted by the reference numerals H and L in FIG. 9. By varying the overlap time and separation time, the efficiency of the controller can be shown here by way of example. Two pulses with a projectile collision speed approximately in the range L arrive respectively at a pulse with a projectile collision speed approximately in the range H. FIG. 9 demonstrates in particular that the collision conditions can be substantially changed from one collision to the next, here with approximately a factor of 3 in the collision speed. The fluctuations within the ranges H and L are in this case unintentional and tolerance-related variations (these are real measured values).

FIG. 10 shows by way of example the control sequence for the valves V1 and V2 in their temporal succession in order to achieve the projectile speed sequences which can be seen in FIG. 9. Different overlaps and separations of pulses relative to one another can be seen.

FIG. 11 sets the succession of the first pulses from FIG. 10 more precisely in terms of time, with the result that a repeating sequence is shown individually here. It can be seen more clearly here that the valve opening times between V1 and V2 change their separation and overlap relatively.

FIG. 12 and FIGS. 13 a) to d) relate to further real laboratory measurements in addition to the above table of values (for FIGS. 4 a) to e)). The measurements were produced with the apparatus described in FIGS. 1 to 3 at a repetition frequency of 10 Hz and a constant supply pressure of 4 bar, wherein the valve opening times for both valves 1 and 2 were constant aT14 ms. In the four measurement points indicated in FIG. 12, which correspond to the following figures in the order from left to right, the overlap time denoted by the symbol δ in FIGS. 13 a) to d) was 6 ms, 5 ms, 4 ms, and 3 ms, with the result that the “net pulse duration”, illustrated on the horizontal axis in FIG. 12, of the accelerating pneumatic pulse of the first valve V1 (before the overlap time δ) was accordingly from 8 ms to 11 ms.

A strictly monotonically increasing relationship, which also does not come as a surprise, can easily be seen in FIG. 12 on the basis of the dashed interpolation line. The more time (with unchanged pressure) available for the acceleration, the higher the impact speed. The collisions took place approximately between 16 ms and 17 ms, that is to say rather soon after the end of the first valve opening time and the overlap time and during the second valve opening time. In this respect, there was a small braking action of the pneumatic pulse from the second valve in all the illustrated operating states and, in all the illustrated states, served somewhat more than half of the second valve opening time for safe and rapid return of the projectile. The variation of the overlap time has varied the (previous) portion of the first valve opening time which is effective for the acceleration and therefore the collision speed.

The above explanations on the basis of FIGS. 4 and 6 to 13 relate to the apparatus illustrated in FIGS. 1 to 3. They can also be transferred to other apparatuses and dimensions on the basis of simple estimates for the projectile movement. In particular, the reversal points of the projectile movement are easily accessible, for example, via the mentioned measuring coil, possibly an analog measuring coil at the distal end of the movement path or via the detection of the collisions by microphone. On this basis, meaningful estimates can be made on the basis of the above descriptions.

Alternatively, the following procedure can be adopted: a desired operating frequency and a desired supply pressure for the two valves are predefined and, for example, it is also predefined that the two valves open for a constant duration, for example for 40% of the reciprocal of the predefined frequency. The controller can then be set up such that the valves open and close precisely in phase at a starting time. In this state, stable movement will not occur because pressure is applied to the projectile on both sides at the same time or pressure is not applied to it from any side. On this basis, it is then possible to change the offset between opening times in both directions in steps, i.e., to open (and close) the second valve in steps somewhat earlier or somewhat later than the first valve. Starting from a certain time offset, i.e., so to speak, starting from a certain phase shift, a stable vibration state of the projectile will occur, which can be established, for example, with the mentioned microphone determination of the collisions at the two ends of the movement path. In addition, it is then possible to determine the intensity of the collision with the applicator and to consider the described phase shift to some extent as a control parameter for the intensity. In this form, a calibration curve can be determined.

In addition, it is of course possible to maintain the phase offset constant in the case of a certain vibration state determined in this form and to change the first and/or the second valve opening duration in steps.

In the individual case, it could occur that a sufficient pressure was not predefined for the desired frequency, i.e., no vibration state with collisions at the ends of the movement path arises even in the case of “anti-phase” controlling of the two valves. It is then accordingly necessary either to increase the pressure somewhat or to reduce the frequency.

Analogously, it is of course also possible to approach suitable operating states empirically in another form. Finally, it is of course possible to simulate the movement behavior of the projectile at least approximately computationally, and empirical tests can then be undertaken on the basis of the results of such simulations.

1. An apparatus for treatment of the human or animal body with mechanical pressure waves, the apparatus comprising:
a projectile guided in the apparatus along a movement path,
an applicator at one end of the movement path,
pneumatic means for application of pneumatic pressure to the projectile for the purpose of movement along the movement path,
wherein the projectile is adapted for striking onto the applicator for generating the mechanical pressure waves,
which pneumatic means has a double valve means for application of pneumatic pressure to the projectile in the direction towards the applicator during a first activation time and for application of pneumatic pressure to the projectile in the reverse direction during a second activation time and a control means for controlling the double valve means,
wherein the apparatus is adapted to allow the first and the second activation time to overlap in an overlap time.

2. The apparatus according to claim 1, in which the double valve means has a first valve for application of pneumatic pressure to the projectile in the direction towards the applicator and a second valve for application of pneumatic pressure to the projectile in the reverse direction, which valves can preferably be controlled independently of one another by the control means.

3. The apparatus according to claim 1, in which the double valve means has a “combination valve” which, depending on the control by the control means, assumes a first switching state for application of pneumatic pressure to the projectile in the direction towards the applicator or a second switching state for application of pneumatic pressure to the projectile in the reverse direction, wherein in each of these switching states the pneumatic connection used in the respectively other switching state for application of pneumatic pressure to the projectile is ventilated by the combination valve or, depending on the control by the control means, assumes a third switching state in which both pneumatic connections used for application of pneumatic pressure to the projectile are applied with pneumatic pressure by the combination valve.

4. The apparatus according to claim 2, in which at least one of the two valves is a two-way valve which applies pneumatic pressure to a pneumatic volume between itself and the projectile in a first switching position during the respective activation time for application of pneumatic pressure to the projectile and which ventilates this pneumatic volume in a second switching position.

5. The apparatus according to claim 1, adapted to control a collision speed of a collision between the projectile and the applicator with the control means by varying the overlap time.

6. The apparatus according to claim 1, in which, in the case of an overlap time at the end of a first activation time, the second activation time is started during a forward movement of the projectile and/or, in the case of an overlap time at the end of a second activation time, the first activation time following this second activation time is started during a return movement of the projectile.

7. The apparatus according to claim 1, adapted to end the first activation time during the second activation time or vice versa.

8. The apparatus according to claim 1, adapted to control an impact speed of the projectile upon impact onto the applicator by means of the portion of the first activation time outside the overlap time associated therewith.

9. The apparatus according to claim 8, wherein in comparison between at least two control states with different periods of overlap of the first and the second activation time, the earlier one of these two activation times is of constant length.

10. The apparatus according to claim 8, wherein in comparison between at least two control states with different periods of overlap, the later one of these two activation times is of variable length.

11. The apparatus according to claim 8, wherein the control means is adapted to vary in different control states a separation time between the first and the second activation time.

12. The apparatus according to claim 1, wherein the pneumatic means comprises a pneumatic compressor, wherein the apparatus is adapted to allow the compressor in the activated state to run at different control states with different impact speeds of the projectile at the same rotational frequency, preferably in principle in the activated state to run at always the same rotational frequency.

13. The apparatus according to claim 1, wherein the projectile can be moved with an impact pulse of between 2 gm/s and 300 gm/s upon impact onto the applicator.

14. The apparatus according to claim 1, adapted to vary, in an iterative operating state with directly successive forward movements of the projectile for impact onto the applicator and return movements, the impact speed of the combined forward and return movement from one to the next such combined forward and return movement.

15. The apparatus according to claim 1, having a measuring means for detecting a passage of the projectile at a point of the movement path, which measuring means is coupled to the control means.
